(19) United States
(12) Patent Application Publication
(10) Pub. No.: US 2014/0276066 A1
(43) Pub. Date: Sep. 18, 2014

(54) IMAGING APPARATUS WITH REINFORCED ELECTRICAL SIGNAL TRANSMISSION MEMBER AND METHOD OF USE THEREOF

(71) Applicant: VOLCANO CORPORATION, San Diego, CA (US)

(72) Inventors: David Caudle, San Diego, CA (US); Lisa Fong, La Jolla, CA (US)

(73) Assignee: VOLCANO CORPORATION, San Diego, CA (US)

(21) Appl. No.: 14/205,797

(22) Filed: Mar. 12, 2014

Related U.S. Application Data
(60) Provisional application No. 61/777,849, filed on Mar. 12, 2013.

(51) Int. Cl. A61B 8/00; A61B 8/12; A61B 5/00

(52) U.S. Cl. CPC A61B 8/56; A61B 5/0066; A61B 8/12; A61B 8/4488; A61B 8/4461
USPC 600/445; 600/407; 600/443; 600/447

(57) ABSTRACT

The invention generally relates to an imaging apparatus having a reinforced electrical signal transmission member, and method of use thereof. In certain aspects, the apparatus includes a hollow elongate body. The apparatus also includes an elongate electrical signal transmission member within the body. The elongate signal transmission member includes two or more outer jacket layers. There is an imaging device within the body and the signal transmission member is coupled to the imaging device.

### RELATED APPLICATION

The present application claims the benefit of and priority to U.S. provisional patent application Ser. No. 61/777,849, filed Mar. 12, 2013, the content of which is incorporated by reference herein in its entirety.

### FIELD OF THE INVENTION

The invention generally relates to an imaging apparatus having a reinforced electrical signal transmission member, and method of use thereof.

### BACKGROUND

Intravascular Ultrasound (IVUS) is an important interventional diagnostic procedure for imaging atherosclerosis and other vessel diseases and defects. In the procedure, an IVUS catheter is threaded over a guidewire into a blood vessel, and images are acquired of the atherosclerotic plaque and surrounding area using ultrasonic echoes. That information is much more descriptive than information from other imaging techniques, such as angiography, which shows only a two-dimensional shadow of a vessel lumen.

There are two types of IVUS catheters commonly in use, mechanical/rotational IVUS catheters and solid state catheters. A solid state catheter (or phased array) has no rotating parts, but instead includes an array of transducer elements (for example 64 elements). In a rotational IVUS catheter, a single transducer having a piezoelectric crystal is rapidly rotated (e.g., at approximately 1800 revolutions per minute) while the transducer is intermittently excited with an electrical pulse. The excitation pulse causes the transducer to vibrate, sending out a series of transmit pulses. The transmit pulses are sent at a frequency that allows time for receipt of echo signals. The sequence of transmit pulses interspersed with receipt signals provides the ultrasound data required to reconstruct a complete cross-sectional image of a vessel.

Typically, the transducer is disposed at a distal end of the IVUS catheter. A coaxial cable is disposed within the catheter and couples to the transducer. The coaxial cable delivers the intermittent electrical transmit pulses to the transducer, and delivers the received electrical echo signals from the transducer to the receiver amplifier. The IVUS catheter is removably coupled to an interface module, which controls the transmitter and receiver circuitry for the transducer.

Maintaining the coaxial cable in a relaxed state is important for producing a high quality image. A problem with IVUS catheters is that the coaxial cable becomes stressed during operation due movement of the catheter through the vasculature it the area of a vessel that needs to be imaged. Stress on the coaxial cable results in poor data acquisition or loss of data, both of which lead to poor image quality.

### SUMMARY

The invention generally provides rotational imaging apparatuses that are configured to prevent kinking or winding of a signal transmission member in the apparatus. Aspects of the invention are accomplished using reinforced signal transmission members. A typical signal transmission member includes only a single outer jacket. The outer jacket is not strong enough to prevent stress on the signal transmission member as an intravascular (IVUS) catheter is advanced through the vasculature to an area of a vessel that needs to be imaged. By reinforcing the signal transmission with additional outer jackets so that (e.g., 2 or more outer jackets), the signal transmission member is strengthened and stress on the signal transmission member is reduced or eliminated. Maintaining the electrical signal transmission member in a relaxed state prevents loss of data or acquisition of poor data, thus leading to production of high quality images.

Apparatuses of the invention include a hollow elongate body. The apparatus also includes an elongate electrical signal transmission member within the body. The elongate signal transmission member includes two or more outer jacket layers. There is an imaging device within the body and the signal transmission member is coupled to the imaging device.

The apparatus may be any apparatus that is used for intravascular imaging and requires a signal transmission member. In certain embodiments, the imaging apparatus is an intravascular ultrasound apparatus. The IVUS catheter apparatus may be a solid state catheter (or phased array) having no rotating parts, but instead includes an array of transducer elements (for example 64 elements). Alternatively, the IVUS catheter may be a rotational IVUS catheter having a single transducer that is rapidly rotated (e.g., at approximately 1800 revolutions per minute). In such embodiments, the imaging apparatus includes an elongate drive member within the body that rotates the transducer. The signal transmission member is disposed within the drive member and also rotates. Both the drive member and the signal transduction member are coupled to the transducer.

Typically, ultrasound systems rely on conventional piezoelectric transducers, built from piezoelectric ceramic (commonly referred to as the crystal) and covered by one or more matching layers (typically thin layers of epoxy composites or polymers). Two advanced transducer technologies that have shown promise for replacing conventional piezoelectric devices are the PMUT (Piezoelectric Micromachined Ultrasonic Transducer) and CMUT (Capacitive Micromachined Ultrasonic Transducer). PMUT and CMUT transducers may provide improved image quality over that provided by the conventional piezoelectric transducer.

Generally, the apparatus may connect to an interface module via the connector. The interface module typically includes components necessary to control the transmitter and receiver circuitry for the transducer. In embodiments of a rotational device, the interface module typically includes components necessary for rotating the drive member and the electrical signal transmission member. Apparatuses of the invention are configured from insertion in a vessel lumen, and include additional features that facilitate operation within the vessel. For example, a distal end of the body may include an atraumatic tip. The atraumatic tip is configured to guide the apparatus through the vessel lumen while avoiding perforation of the lumen. Additionally, the body, the drive member, and/or the signal transmission member may be flexible so that the apparatus may more easily be advanced through the vessel.

Other aspects of the invention provide methods for imaging a vessel lumen. Such methods involve providing an imaging apparatus that includes a hollow elongate body, an elongate electrical signal transmission member within the body, in which the elongate signal transmission member comprises two or more outer jacket layers, and an imaging device within the body, in which the signal transmission member is coupled to the imaging device. The apparatus is inserted into a vessel lumen and used to obtain image data of the vessel lumen.

### BRIEF DESCRIPTION OF THE DRAWINGS

FIG. 1 is a simplified fragmentary diagrammatic view of a rotational IVUS probe.

FIG. 2 is a simplified fragmentary diagrammatic view of an interface module and catheter for the rotational IVUS probe of FIG. 1 incorporating basic ultrasound transducer technology.

FIG. 3 shows a prior art coaxial cable having only a single outer jacket.

FIG. 4 is a cross section of a signal transmission member built with two outer jacket layers.

### DETAILED DESCRIPTION

The invention generally relates to an imaging apparatus having a reinforced electrical signal transmission member, and method of use thereof. Apparatuses of the invention include a hollow elongate body. The apparatus also includes an elongate electrical signal transmission member within the body. The elongate signal transmission member includes two or more outer jacket layers. There is an imaging device within the body and the signal transmission member is coupled to the imaging device.

Typically, apparatuses of the invention are provided in the form of a catheter. Any imaging device known in the art may be used with apparatuses of the invention. Exemplary devices include ultrasound devices and optical coherence tomography (OCT) devices. In particular embodiments, the imaging device is an ultrasound device and apparatuses of the invention are intravascular ultrasound (IVUS) catheters. The general design and construction of such catheters is shown, for example in Yock, U.S. Pat. Nos. 4,794,931, 5,000,185, and 5,313,949; Sieben et al., U.S. Pat. Nos. 5,243,988, and 5,353,798; Crowley et al., U.S. Pat. No. 4,951,677; Pomeranz, U.S. Pat. No. 5,095,911, Griffith et al., U.S. Pat. No. 4,841,977, Maroney et al., U.S. Pat. No. 5,373,849, Born et al., U.S. Pat. No. 5,176,141, Lancee et al., U.S. Pat. No. 5,240,003, Lancee et al., U.S. Pat. No. 5,375,602, Gardineer et at., U.S. Pat. No. 5,373,845, Seward et al., Mayo Clinic Proceedings 71(7):629-635 (1996), Packer et al., Cardiostim Conference 833 (1994), “Ultrasound Cardioscopy,” Eur. J.C.P.E. 4(2):193 (June 1994), Eberle et al., U.S. Pat. No. 5,453,575, Eberle et al., U.S. Pat. No. 5,368,037, Eberle et at., U.S. Pat. No. 5,183,048, Eberle et al., U.S. Pat. No. 5,167,233, Eberle et at., U.S. Pat. No. 4,917,097, Eberle et at., U.S. Pat. No. 5,135,486, and other references well known in the art relating to intraluminal ultrasound devices and modalities. The catheter will typically have proximal and distal regions, and will include an imaging tip located in the distal region. Such catheters have an ability to obtain echographic images of the area surrounding the imaging tip when located in a region of interest inside the body of a patient. The catheter, and its associated electronic circuitry, will also be capable of defining the position of the catheter axis with respect to each echographic data set obtained in the region of interest.

Besides intravascular ultrasound, other types of ultrasound catheters can be made using the teachings provided herein. By way of example and not limitation, other suitable types of catheters include non-intravascular intraluminal ultrasound catheters, intracardiac echo catheters, laparoscopic, and interstitial catheters. In addition, the probe may be used in any suitable anatomy, including, but not limited to, coronary, carotid, neuro, peripheral, or venous.

In certain embodiments, the ultrasound apparatus is a rotational ultrasound apparatus. FIG. 1 shows a rotational intravascular ultrasound probe 100 for insertion into a patient for diagnostic imaging. The probe 100 includes a catheter 101 having a catheter body 102 and a hollow transducer shaft 104. The catheter body 102 is flexible and has both a proximal end portion 106 and a distal end portion 108. The catheter body 102 may be a single lumen polymer extrusion, for example, made of polyethylene (PE), although other polymers may be used. Further, the catheter body 102 may be formed of multiple grades of PE, for example, HDPE and LDPE, such that the proximal portion exhibits a higher degree of stiffness relative to the mid and distal portions of the catheter body. This configuration provides an operator with catheter handling properties required to efficiently perform the desired procedures.

The catheter body 102 is a sheath surrounding the transducer shaft 104. For explanatory purposes, the catheter body 102 in FIG. 1 is illustrated as visually transparent such that the transducer shaft 104 disposed therein can be seen, although it will be appreciated that the catheter body 102 may or may not be visually transparent. The transducer shaft 104 is flushed with a sterile fluid, such as saline, within the catheter body 102. A fluid injection port (not shown) may be supplied at a junction of the catheter body 102 to the interface module so that the space inside the catheter body 102 can be flushed initially and periodically. The fluid serves to eliminate the presence of air pockets around the transducer shaft 104 that adversely affect image quality. The fluid can also act as a lubricant. The transducer shaft 104 has a proximal end portion 110 disposed within the proximal end portion 106 of the catheter body 102 and a distal end portion 112 disposed within the distal end portion 108 of the catheter body 102.

The distal end portion 108 of the catheter body 102 and the distal end portion 112 of the transducer shaft 104 are inserted into a patient during the operation of the probe 100. The usable length of the probe 100 (the portion that can be inserted into a patient) can be any suitable length and can be varied depending upon the application. The distal end portion 112 of the transducer shaft 104 includes a transducer subassembly 118.

The transducer subassembly 118 is used to obtain ultrasound information from within a vessel. It will be appreciated that any suitable frequency and any suitable quantity of frequencies may be used. Exemplary frequencies range from about 5 MHz to about 80 MHz. Generally, lower frequency information (e.g., less than 40 MHz) facilitates a tissue versus blood classification scheme due to the strong frequency dependence of the backscatter coefficient of the blood. Higher frequency information (e.g., greater than 40 MHz) generally provides better resolution at the expense of poor differentiation between blood speckle and tissue, which can make it difficult to identify the lumen border. Blood speckle reduction algorithms such as motion algorithms (such as ChromaFlo, Q-Flow, etc.), temporal algorithms, harmonic signal processing, can be used to enhance images where light back scattered from blood is a problem.

The proximal end portion 106 of the catheter body 102 and the proximal end portion 110 of the transducer shaft 104 are connected to an interface module 114 (sometimes referred to as a patient interface module or PIM). The proximal end portions 106, 110 are fitted with a catheter hub 116 that is removably connected to the interface module 114.

The catheter body 102 may include a flexible atraumatic distal tip. For example, an integrated distal tip can increase the safety of the catheter by eliminating the joint between the distal tip and the catheter body. The integral tip can provide a smoother inner diameter for ease of tissue movement into a collection chamber in the tip. During manufacturing, the transition from the housing to the flexible distal tip can be finished with a polymer laminate over the material housing. No weld, crimp, or screw joint is usually required. The atraumatic distal tip permits advancing the catheter distally through the blood vessel or other body lumen while reducing any damage caused to the body lumen by the catheter. Typically, the distal tip will have a guidewire channel to permit the catheter to be guided to the target lesion over a guidewire. In some exemplary configurations, the atraumatic distal tip includes a coil. In some configurations the distal tip has a rounded, blunt distal end. The catheter body can be tubular and have a forward-facing circular aperture which communicates with the atraumatic tip.

The rotation of the transducer shaft 104 within the catheter body 102 is controlled by the interface module 114, which provides a plurality of user interface controls that can be manipulated by a user. The interface module 114 also communicates with the transducer subassembly 118 by sending and receiving electrical signals to and from the transducer subassembly 118 via at least one electrical signal transmission member (e.g., wires or coaxial cable) within the transducer shaft 104. The interface module 114 can receive, analyze, and/or display information received through the transducer shaft 104. It will be appreciated that any suitable functionality, controls, information processing and analysis, and display can be incorporated into the interface module 114. Further description of the interface module is provided, for example in Corl (U.S. patent application number 2010/0234736).

The transducer shaft 104 includes a transducer subassembly 118, a transducer housing 120, and a drive member 122. The transducer subassembly 118 is coupled to the transducer housing 120. The transducer housing 120 is attached to the drive member 122 at the distal end portion 112 of the transducer shaft 104. The drive member 122 is rotated within the catheter body 102 via the interface module 114 to rotate the transducer housing 120 and the transducer subassembly 118. The transducer subassembly 118 can be of any suitable type, including but not limited to one or more advanced transducer technologies such as PMUT or CMUT. The transducer subassembly 118 can include either a single transducer or an array.

FIG. 2 shows a rotational IVUS probe 200 utilizing a common spinning element 232. The probe 200 has a catheter 201 with a catheter body 202 and a transducer shaft 204. As shown, the catheter hub 216 is near the proximal end portion 206 of the catheter body 202 and the proximal end portion 210 of the transducer shaft 204. The catheter hub 216 includes a stationary hub housing 224, a dog 226, a connector 228, and bearings 230. The dog 226 mates with a spinning element 232 for alignment of the hub 216 with the interface module 214 and torque transmission to the transducer shaft 204. The dog 226 rotates within the hub housing 224 utilizing the bearings 230. The connector 228 in this figure is coaxial. The connector 228 rotates with the spinning element 232, described further herein.

As shown, the interior of the interface module 214 includes a motor 236, a motor shaft 238, a printed circuit board (PCB) 240, the spinning element 232, and any other suitable components for the operation of the IVUS probe 200. The motor 236 is connected to the motor shaft 238 to rotate the spinning element 232. The printed circuit board 240 can have any suitable number and type of electronic components 242, including but not limited to the transmitter and the receiver for the transducer.

The spinning element 232 has a complimentary connector 244 for mating with the connector 228 on the catheter hub 216. As shown, the spinning element 232 is coupled to a rotary portion 248 of a rotary transformer 246. The rotary portion 248 of the transformer 246 passes the signals to and from a stationary portion 250 of the transformer 246. The stationary portion 250 of the transformer 246 is wired to the transmitter and receiver circuitry on the printed circuit board 240.

The transformer includes an insulating wire that is layered into an annular groove to form a two- or three-turn winding. Each of the rotary portion 250 and the stationary portion 248 has a set of windings, such as 251 and 252 respectively. Transformer performance can be improved through both minimizing the gap between the stationary portion 250 and the rotary portion 248 of the transformer 246 and also by placing the windings 251, 252 as close as possible to each other.

A problem that occurs during operation of IVUS catheters is that the signal transmission member tends to become kinked it is not strong enough to handle the tortious nature of the vasculature. That causes stress on the signal transmission member, which leads to acquisition of poor image data or loss of data. FIG. 3 a typically prior art signal transmission member. Prior art cables only include a single outer jacket layer, which limits their strength.

That problem is solved by providing at least one additional outer jacket layer on the signal transmission member. FIG. 4 shows an exemplary embodiment of a signal transmission member having two outer jacket layers. Accordingly, the signal transmission member will have at least two outer jacket layers, at least three, at least four, at least five, at least 10, at least 15 outer jacket layers. Such additional layers reinforces and stabilizes the electrical signal transmission member, thereby preventing the electrical signal transmission member from becoming stressed. By using extra outer layers, signal transmission members can be produced that do not use twisted quad cable or cable in which the center conductor wire are twisted, as shown in FIG. 4.

The extra outer jacket layers may be applied by any methods known in the art. For example, an extra layer of Mylar may be wrapped around the signal transmission member. In certain embodiments, an adhesive such as an epoxy is used to attach the extra outer jacket layer to the signal transmission member. Other adhesives include thermo-setting plastics, UV curable adhesives, or silicone rubber gels.

### Incorporation By Reference

References and citations to other documents, such as patents, patent applications, patent publications, journals, books, papers, web contents, have been made throughout this disclosure. All such documents are hereby incorporated herein by reference in their entirety for all purposes.

### Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. An imaging apparatus, the apparatus comprising:
a hollow elongate body;
an elongate electrical signal transmission member within the body, wherein the elongate signal transmission member comprises two or more outer jacket layers; and
an imaging device within the body, wherein the signal transmission member is coupled to the imaging device.

2. The apparatus according to claim 1, wherein the imaging device is an ultrasound device.

3. The apparatus according to claim 2, wherein the ultrasound device comprises an array of stationary ultrasound transducers.

4. The apparatus according to claim 2, wherein the ultrasound device is a rotational ultrasound device.

5. The apparatus according to claim 4, further comprising a rotatable elongate drive member within the body, wherein the elongate electrical signal transmission is rotatable and disposed within the drive member.

6. The apparatus according to claim 5, wherein the apparatus connects to an interface module via a connector at a proximal end of the body, the interface module comprising components to rotate the drive member and the electrical signal transmission member.

7. The apparatus according to claim 6, wherein the imaging device is coupled to a distal end of the drive member.

8. The apparatus according to claim 1, wherein the electrical signal transmission member is coaxial cable.

9. A method of obtaining image data of a vessel lumen, the method comprising:
providing an imaging apparatus that comprises a hollow elongate body; an elongate electrical signal transmission member within the body, wherein the elongate signal transmission member comprises two or more outer jacket layers; and an imaging device within the body, wherein the signal transmission member is coupled to the imaging device; and
using the apparatus to obtain image data from within a vessel.

10. The method according to claim 9, wherein the imaging device is an ultrasound device.

11. The method according to claim 10, wherein the ultrasound device comprises an array of stationary ultrasound transducers.

12. The method according to claim 10, wherein the ultrasound device is a rotational ultrasound device.

13. The method according to claim 12, further comprising a rotatable elongate drive member within the body, wherein the elongate electrical signal transmission is rotatable and disposed within the drive member.

14. The method according to claim 13, wherein the apparatus connects to an interface module via a connector at a proximal end of the body, the interface module comprising components to rotate the drive member and the electrical signal transmission member.

15. The method according to claim 14, wherein the imaging device is coupled to a distal end of the drive member.

16. The method according to claim 15, wherein the electrical signal transmission member is coaxial cable.
